(19) United States
(12) Patent Application Publication
(10) Pub. No.: US 2009/0054790 A1
(43) Pub. Date: Feb. 26, 2009

(54) Method and Arrangement for Generating a Signal which Corresponds to the Degree of Opening of the Vocal Folds of the Larynx

(76) Inventor: Jurgen Czaniera, Bonndorf (DE)

Correspondence Address: THE NATH LAW GROUP, 112 South West Street, Alexandria, VA, 22314, US

(21) Appl. No.: 12/222,456

(22) Filed: Aug. 8, 2008

(30) Foreign Application Priority Data
Aug. 16, 2007 (DE) 102007038859.6

(51) Int. Cl. A61B 1/06; A61B 6/00

(52) U.S. Cl. 600/476

(57) ABSTRACT

The invention relates to a method for generating a signal which corresponds to the degree of opening of the vocal folds of the larynx, in which the vocal folds are illuminated by means of a light source and are optically observed by means of an endoscope and a signal which is modulated by the movement of the vocal folds, the vocal fold signal, is displayed and/or recorded, wherein the vocal folds are illuminated with light having a wavelength greater than 600 nm or with red light, and wherein the light which is modulated by the movement of the vocal folds, the vocal fold light, is received by a sensor and is detected in order to generate a vocal fold signal which can be visualised, and visible light which contains little infrared or red light is used at the same time to observe the vocal folds.

The invention relates to a method for generating a signal which corresponds to the degree of opening of the vocal folds of the larynx, according to the preamble of Claim 1, and also to arrangements for carrying out this method.

The subsequently published document DE 10 2006 008 990 A1 proposes a method in which the vocal folds are illuminated on the one hand with light in the near-infrared light range (NIR light) and on the other hand with visible light which is free of infrared light. In said document, the NIR light is modulated in terms of its intensity by the movement of the vocal folds, is received by a sensor and is detected in order to generate a vocal fold signal which can be visualised. In order to allow a simultaneous endoscopic observation of the vocal folds, without impairing the vocal fold signal, light which is free of infrared light is used so that the vocal fold signal can be separated from the image signal by simple means.

The use of light in the near-infrared range is advantageous because organic tissue is particularly transparent to this light. The further away the infrared light, the less it penetrates due to the H₂O absorption of the tissue. The penetration becomes increasingly poor in the direction of the visible wavelengths, so that a limit is reached at a wavelength of 600 nm. However, this means that the method according to DE 10 2006 008 990 A1 in principle still functions even with longer-wave light, although it is more difficult to distinguish the light which is modulated by the vocal folds, the vocal fold light, from the illumination.

The present invention proposes measures which can be used to implement the method defined generally in Claim 1.

As specified in Claim 1, the invention can in principle be carried out in a method according to the preamble if the vocal folds are illuminated with light having a wavelength greater than 600 nm or with red light, wherein the light which is modulated by the movement of the vocal folds, the vocal fold light, is received by a sensor and is detected in order to generate a vocal fold signal which can be visualised, and if visible light which at least contains little infrared or red light is used at the same time to observe the vocal folds.

Suitable sensors include in principle all components or modules which convert light signals into electric signals, such as e.g. photodiodes or photoresistors, but also linear image sensors or two-dimensional image sensors, as used in video cameras.

According to the proposal of Claim 2, in order to suppress interfering signals, the vocal fold light is modulated and is synchronously demodulated after receipt by the sensor. It should be ensured here that the modulation frequency is much greater than the base frequency of the vocal fold signal, which is approximately 1 kHz.

According to Claim 3, therefore, a modulation frequency of at least 20 kHz is proposed. The resolution of the vocal fold signal improves as the modulation frequency increases, wherein the complexity is quite high for modulation frequencies greater than 100 kHz.

In order to accurately observe the movement sequences of the vocal folds, it is proposed in DE 10 2006 008 990 A1 to use for illuminating the vocal folds a stroboscopic light source which is triggered by means of the vocal fold signal.

According to the proposal in Claim 4 of this patent application, the same result can be achieved according to the invention if a video camera is used, the camera shutter of which is triggered by means of the vocal fold signal. In this case, the vocal folds are illuminated with constant light instead of with flashing light.

This approach opens up a further possibility which is proposed in Claim 5, according to which continuous light which contains infrared light is used to illuminate the vocal folds and there is no separate emitter for producing the NIR light.

As proposed in Claim 10 of DE 10 2006 008 990 A1, for this purpose the sensor for receiving the vocal fold signal must be attached to the neck of the patient below the larynx. The vocal folds are illuminated with continuous light which also contains infrared light components, wherein the shutter of the camera is triggered by the red or infrared light component received by the sensor. Nevertheless it must be acknowledged that, in this approach, extraneous radiation, e.g. from lamps, may lead to interference. However, this can be compensated if necessary by modulating the endoscopic illumination with a frequency which is too high for the camera, so that the latter receives effectively a continuous light. The signal can be reconstructed through demodulation. Also when using stroboscopic light sources, it is difficult to generate a useful vocal fold signal since the output signal of the sensor has to be extremely highly amplified.

The signal generated by the flash of the stroboscopic light source is large enough that the sensor, although it is in fact optimised only for infrared light, also reacts to visible light. Furthermore, even LEDs generate a certain amount, albeit small, of infrared light. Therefore, despite all the filters and selective mirrors, the light generated by the flash interferes with the useful signal. Although this is not disadvantageous, since the time of the flash can be seen in the useful signal, which is also informative, it nevertheless has the disadvantage that it can no longer be triggered by means of the useful signal.

In order to avoid this disadvantage, two different measures are proposed.

According to the proposal in Claim 6, interference in the signal received by the sensor is compensated by electronic means, namely by means of a track-and-hold amplifier which is controlled by the stroboscopic flash.

According to the proposal in Claim 7, light signals of the stroboscopic light source, which are therefore free of useful signals, are coupled out by means of a sensor, wherein the signals generated by the sensor are superposed on the useful signal in phase opposition thereto in order to compensate interfering signals.

This coupling-out may take place directly at the stroboscopic light source or else light signals may be coupled out via an optical fibre.

An arrangement which is suitable for this method forms the subject matter of Claim 8 and will be explained in detail below on the basis of an example of embodiment.

Compared to the method according to Claim 6, this approach has the advantage that the interfering signal is not passed on but rather is immediately compensated.

In the arrangement specified in Claim 8, a mirror is provided which suppresses, i.e. reflects away, the infrared or red light signals by reflection, but on the other hand allows the visible light signals to pass through.

In principle, however, it is possible and equivalent to use a mirror which allows infrared and red light radiation to pass through and which reflects away the visible light signals. The only important thing is to separate the infrared and red light signals from the visible light signals.

It is better if, as in the arrangement according to Claim 8, the interfering signal is not passed on at all, but rather is immediately compensated. This proposal will be explained in detail below on the basis of an example of embodiment.

A further variant of an arrangement for carrying out the method according to the invention forms the subject matter of Claim 9, in which a video camera is provided for observing the vocal folds.

While according to Claim 7 of DE 10 2006 008 990 A1 the mirror or the prism is arranged in the video camera behind the camera lens, this proposal can also be achieved if, as in the proposal according to the invention in Claim 8, the mirror or the prism is arranged in front of the camera in such a way that the vocal fold signal passed through the endoscope lens is coupled out by reflection and is supplied to the sensor, while the visible light is supplied to the image sensor of the video camera.

The subject matter of the invention will be explained in detail below on the basis of an example of embodiment.

### IN THE DRAWING

FIG. 1 shows a schematic view of an arrangement according to the invention with the head of the observer and the head of the patient, and

FIG. 2 shows an enlarged detail from FIG. 1.

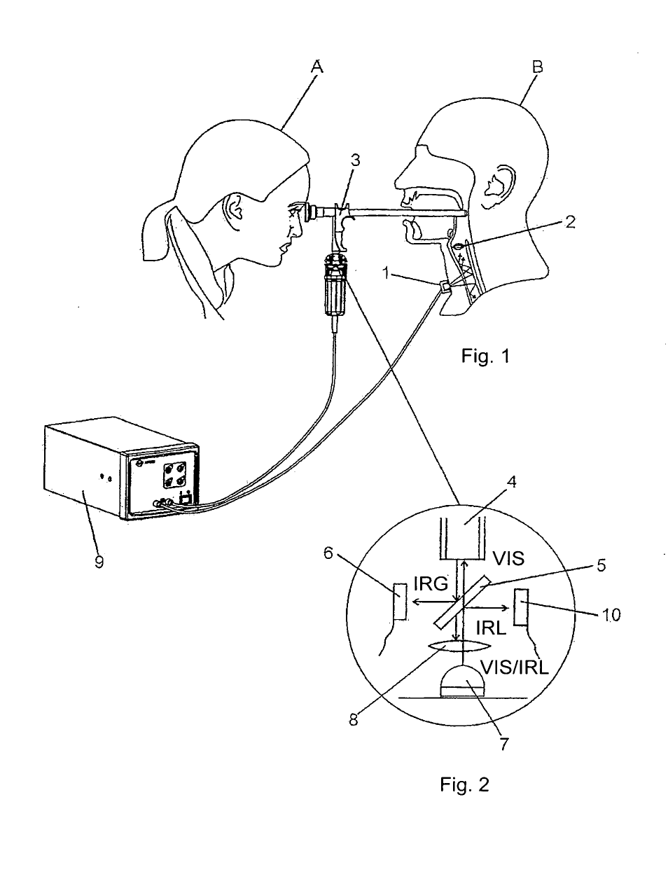

As in the example of embodiment of the invention shown in FIGS. 1 and 2 of DE 10 2006 008 990 A1, the illustrated arrangement consists essentially of an endoscope 3 which is introduced into the pharyngeal space of the patient B and by means of which the observer A, that is to say e.g. the physician, can optically observe the vocal folds 2. Flashing light produced by a stroboscopic light source 7 is fed into the illumination input 4 of the endoscope. In order that this light is free of infrared or red light, an infrared light mirror 5 is arranged between the light source 7 and the illumination input 4 and reflects, i.e. couples out, any infrared light contained in the light of the light source 7 and allows only light in the visible wavelength range to pass through.

An emitter 1 which generates light in the near-infrared range, namely NIR light, is provided below the larynx of the patient B. The light produced by said emitter passes through the tissue of the neck and illuminates the trachea and thus the vocal folds 2 from below. This light passes through the vocal folds 2 to a greater or lesser extent depending on the degree of opening, and thus reaches the endoscope 3. It runs through the illumination optical fibre of the endoscope 3 and, via the illumination input 4, reaches the infrared light mirror 5. The latter reflects the infrared light IRG emerging from the illumination input 4 of the endoscope 3 onto the infrared light sensor 6. Since this infrared light IRG is modulated in terms of its intensity by the movement of the vocal fold opening, the glottis, the sensor 6 generates a signal which corresponds to the movement of the vocal folds.

Unlike in the arrangement shown in FIG. 4 of DE 10 2006 008 990 A1, a second identical sensor 10 is arranged opposite the sensor 6, which second sensor receives the flashing light with parasitic IR signal which is coupled out by means of the mirror 5, while the sensor 6 receives mainly the IR signal with parasitic flashing light. After suitable amplification, the output signal of the sensor 10 is superposed on the output signal of the sensor 6 in phase opposition thereto, as a result of which the interference caused by the flash is compensated. This signal is supplied to the evaluation unit 9, which is triggered by the vocal fold signals.

### LIST OF REFERENCES

1 emitter
2 vocal folds
3 endoscope
4 illumination input
5 infrared light mirror
6 sensor
7 light source
8 lens
9 evaluation unit
10 sensor
A observer
B patient

1. Method for generating a signal which corresponds to the degree of opening of the vocal folds of the larynx, in which the vocal folds are illuminated by means of a light source and are optically observed by means of an endoscope and a signal which is modulated by the movement of the vocal folds, the vocal fold signal, is displayed and/or recorded, wherein the vocal folds are illuminated with light having a wavelength greater than 600 nm or with red light, and in that the light which is modulated by the movement of the vocal folds, the vocal fold light, is received by a sensor and is detected in order to generate a vocal fold signal which can be visualised, wherein visible light which contains little infrared or red light is used at the same time to observe the vocal folds.

2. Method according to claim 1, wherein, in order to suppress interfering signals, the light for illuminating the vocal folds is modulated and is synchronously demodulated after receipt by the sensor.

3. Method according to claim 2, wherein the vocal fold light is modulated with a frequency of at least 20 kHz.

4. Method according to claim 1, in which the vocal folds are observed by means of a video camera, wherein the video camera shutter is triggered by means of the vocal fold signal.

5. Method according to claim 4, in which the sensor for receiving the vocal fold signal is attached to the neck of the patient below the larynx, wherein the vocal folds are illuminated with continuous light which also contains infrared light components, wherein the shutter of the camera is triggered by the red or infrared light component received by the sensor.

6. Method according to claim 1, in which the vocal folds are illuminated by means of a stroboscopic light source which is triggered by the movement of the vocal folds, wherein interference in the signal received by the sensor is compensated by means of a track-and-hold amplifier which is controlled by the stroboscopic flash.

7. Method according to claim 1, in which the vocal folds are illuminated by means of a stroboscopic light source which is triggered by the movement of the vocal folds, wherein light signals of the stroboscopic light source are coupled out by means of a sensor and the signals generated by the sensor are superposed on the useful signal in phase opposition thereto in order to compensate interferign signals.

8. Arrangement for carrying out the method according to claim 7, comprising an emitter which can be attached to the neck of a patient below the larynx and which serves to illuminate the vocal folds, and an endoscope which can be introduced into the pharynx of the patient and which serves to receive the vocal fold signals, to supply stroboscopic light which contains little infrared or red light and to observe the vocal folds, wherein the vocal fold light on the light source or camera side is coupled out by means of a mirror or prism which is transparent to the light containing little infrared or red light but on the other hand reflects NIR and red light, and is supplied to the sensor which is arranged between the stroboscopic light source and the illumination input of the endoscope lens in such a way that, on the one hand, infrared or red light radiation contained in the light of the light source is suppressed by reflection and, on the other hand, the vocal fold signal passed through the endoscope lens is coupled out by reflection and is supplied to the sensor, wherein a second identical sensor (10) is arranged opposite the sensor (6) on the other side of the reflective mirror (5), and its output signal is matched by amplification to the output signal of the first sensor (6) and is superposed on the latter in phase opposition in such a way that interference caused by stroboscopic flashes is compensated.

9. Arrangement for carrying out the method according to claim 7, in which an emitter which can be attached to the neck of a patient below the larynx serves to illuminate the vocal folds, and an endoscope which can be introduced into the pharynx of the patient and which serves to receive the vocal fold signals, to supply light which contains little red or infrared light and to observe the vocal folds, the light for illuminating the vocal folds is passes through the lens of the endoscope and on the light source or camera side is coupled out by means of a mirror or prism which is transparent to the light containing little red or infrared light but on the other hand reflects the vocal fold light, and is supplied to the sensor, wherein the mirror or the prism is arranged in front of the lens of the video camera connected to the endoscope, and in that the vocal fold signal passed through the endoscope, and in that the vocal fold signal passed through the endoscope lens is coupled out by reflection and is supplied to the sensor, while the visible light is supplied to the image sensor of the video camera.
